(19) United States
(12) Patent Application Publication
(10) Pub. No.: US 2009/0306772 A1
(43) Pub. Date: Dec. 10, 2009

(54) Ocular Scaffolds and Methods for Subretinal Repair of Bruch's Membrane

(75) Inventors: Sarah L. Tao, Cambridge, MA (US); Henry J. Klassen, Irvine, CA (US); Michael J. Young, Ipswich, MA (US)

Correspondence Address: Casimir Jones, S.C., 2275 DEMING WAY, SUITE 310, MIDDLETON, WI, 53562, US

(73) Assignee: THE REGENTS OF THE UNIVERSITY OF CALIFORNIA, Oakland, CA (US)

(21) Appl. No.: 12/426,066

(22) Filed: Apr. 17, 2009

Related U.S. Application Data
(60) Provisional application No. 61/046,295, filed on Apr. 18, 2008.

(51) Int. Cl. A61F 2/14; C08G 63/08

(52) U.S. Cl. 623/4.1; 528/354; 427/2.24; 977/762

(57) ABSTRACT

The present invention provides ocular scaffolds composed of poly (e-caprolactone) configured to be inserted into the sub-retinal space of a subject, as well as methods for treating eye disease (e.g., age-related macular degeneration) with such scaffolds. The present invention also provides methods of making such scaffolds.

The present application claims priority to U.S. Provisional Application Ser. No. 61/046,295 filed Apr. 18, 2008, which is herein incorporated by reference in its entirety.

### STATEMENT REGARDING FEDERALLY SPONSORED RESEARCH OR DEVELOPMENT

The invention was made with government support under grant T32 EY07145-06 awarded by the National Eye Institute. The government has certain rights in the invention.

### FIELD OF THE INVENTION

The present invention relates to ocular scaffolds composed of poly (e-caprolactone) configured to be inserted, for example, into the sub-retinal space of a subject, as well as methods for treating eye disease (e.g., age-related macular degeneration) with such scaffolds. The present invention also relates to methods of making such scaffolds.

### BACKGROUND OF THE INVENTION

Due to both shifting demographics and advances in other diseases, age-related macular degeneration (AMD) is emerging as an increasingly significant healthcare challenge within the developed world, particularly in countries with large European populations. At present, treatment options are quite limited. Over the last several years, strategies for eliminating abnormal blood vessels under the central retina (macula) have been shown to help a significant proportion of patients with the ‘wet’ form of age-related macular degeneration (AMD). Unfortunately, these vessels often recur because the underlying structural defects in Bruch's membrane are not repaired. In addition, no treatment is available for the restoration of the retinal pigment epithelium (RPE) in patients with the ‘dry’ form of AMD (without abnormal blood vessels), again because of underlying defects in Bruch's membrane that prevent RPE cells from adhering to this structure to reform an intact monolayer. In either case, a method is needed for local restoration of the integrity of Bruch's membrane that will prevent the ingress of neovascular anomalies and/or allow the reconstitution of the RPE monolayer, either by host or grafted cells.

When considering potential restorative approaches to AMD, it is important that the key pathology is effectively addressed. Local submacular repair of Bruch's membrane is therefore fundamental to restoration of the RPE monolayer and preservation of the adjacent photoreceptors (rods and cones) that are essential for vision. Transplantation of cells and tissues might be of additional benefit in relatively advanced cases of AMD once the underlying defect in Bruch's membrane is repaired. Up until now, experimental attempts to repair Bruch's membrane have been frustrated by a number of significant challenges. These challenges include the need for a material that does not induce an inflammatory or foreign body response when implanted beneath the retina and/or RPE, the need for a material construction that allows RPE cells to adhere and grow as an undistorted monolayer while also not disturbing the precise organization of the overlying photoreceptor outer segments, the need for the material to be sufficiently thin and porous for maintaining normal structural relationships in the macula and for diffusion of physiologically important molecules between choroid, RPE, and retina, the need for the material to be resilient with sufficient elasticity and not be overly brittle so that it can be surgically delivered intact to the subretinal space. Another desirable quality is biodegradability.

Previous work has shown that candidate materials generally fall short of many of the above requirements and desirable properties. The heretofore most promising of these, amniotic membrane, is compromised by the strong tendency to scroll up once positioned in the subretinal space. Overcoming this tendency would, in itself, likely require the addition of yet another material that would then have to meet the above requirements/desirable properties as well. What is needed, therefore, is scaffold made of material that meet the above criteria and thus enable the design of a synthetic subretinal implant of value as a medical device, notably for use in maculoplastic therapy in patients with AMD.

### SUMMARY OF THE INVENTION

The present invention provides ocular scaffolds composed of poly (e-caprolactone) configured to be inserted into the sub-retinal space of a subject, as well as methods for treating eye disease (e.g., age-related macular degeneration) with such scaffolds. The present invention also provides methods of making such scaffolds.

In some embodiments, the present invention provides devices comprising a scaffold configured to be inserted into the sub-retinal space of a subject, wherein the scaffold comprises poly(e-caprolactone). In particular embodiments, the present invention provides methods of treating eye-disease, comprising: inserting a scaffold into the subretinal space of a subject, wherein the scaffold comprises poly(e-caprolactone). In further embodiments, the present invention provides methods of making a device for insertion into the sub-retinal space of subject comprising; a) treating poly(e-caprolactone) to generate a scaffold configured to be inserted into the subretinal space of a subject; and b) contacting the scaffold with donor cells, such that the scaffold is at least partially coated by the donor cells.

In certain embodiments, the present invention provides methods of making a device for insertion into the sub-retinal space of subject comprising: contacting a scaffold with donor cells such that said scaffold is at least partially coated by said donor cells, wherein the scaffold is configured to be inserted into the sub-retinal space of a subject and comprises poly(e-caprolactone).

In certain embodiments, the scaffolds of the present invention are present in opthamologically compatible solution or other physiologically buffered solutions. For example, in certain embodiments, the scaffold are in such opthamologically compatible solutions in a container or other packaging such that they can be shipped to surgeon's office for use. Opthamologically compatible solutions are known in the art, such as those used with contact lenses or other products designed to be installed into the eye. In certain embodiments, the opthamological solutions contain one or more antibiotics. In certain embodiments, the present invention provides kits or systems composed of the scaffolds of the present invention in combination with a container and opthamologically compatible solution or physiologically buffered solution.

In some embodiments, the scaffold is formed from nanowires, wherein the nanowires comprise the poly(e-caprolactone). In particular embodiments, the device further comprises donor cells, wherein the scaffold is at least partially coated with the donor cells. In additional embodiments, the donor cells are selected from: RPE cells, stem cells, photoreceptors, precursors, neural or retinal progenitor cells (NPCs and RPCs). In other embodiments, the device further comprises a protein coating (e.g., laminin or similar protein), and wherein the scaffold is coated with the protein coating. In certain embodiments, the scaffold is smooth. In additional embodiments, the scaffold is further configured to serve as a prosthetic Bruch's membrane. In particular embodiments, the scaffold is between about 1.5-6 mm in length (e.g., 1.5 mm . . . 2.0 mm . . . 2.5 mm . . . 3.5 mm . . . 4.5 mm . . . 5.5 . . . 6.0 mm in length), and about 1.5-6 in width (e.g., 1.5 mm . . . 2.0 mm . . . 2.5 mm . . . 3.5 mm . . . 4.5 mm . . . 5.5 . . . 6.0 mm in width). In other embodiments, the scaffold has a thickness of about 3-20 um, or 5-8 um in thickness (e.g., 3.0 um . . . 5.0 um . . . 7.5 um . . . 9.0 um . . . 12 um . . . 15 um . . . 18 um . . . 20 um in thickness). Any combination of the foregoing lengths, widths, and thicknesses may be employed.

In particular embodiments, the insertion leads to a restoration of the retinal pigment epithelium monolayers in the subject. In other embodiments, the scaffold serves as a prosthetic Bruch's membrane. In some embodiments, the scaffold allows the host RPE cells to regenerate the RPE monolayer and serves to block ingress by pathological neovascular structures. In further embodiments, the subject has age-related macular degeneration (AMD).

### DESCRIPTION OF THE DRAWINGS

FIG. 1. Poly(e-caprolactone) (PCL) Nanofiber Fabrication and GFP⁺mRPC Growth. PCL is template-synthesized to form short (˜2.5 μm) fiber length (SNW), long (˜27 μm) fiber length (LNW) and smooth control scaffolds. (a) Proliferation of GFP⁺ mRPCs cultured on short, long, and smooth PCL scaffolds evaluated over seven days. The average numbers of adherent mRPCs at days 1, 3, and 7 on SNW were 6688, 36478, 95542, LNW were 6799, 26044, 118389, and Smooth were 3973, 30217, 83205 respectively. (b,c) Fluorescent micrograph of GFP⁺ mRPCs on LNW scaffolds at day 1 and 7 after initial 24 h adherence periods, respectively. Error bar: Standard Error of Mean, Scale:100 μm

FIG. 2. Scanning Electron Microscopy of mRPCs cultured on SNW, LNW and Smooth PCL Scaffolds. RPCs were seeded and allowed to proliferate for 7 days. (a,b) RPCs develop on the upper edge of aggregated short nanowires extending lamelapodia-like structures towards adjacent cells on day 3 and 7, respectively. (c,d) RPCs seeded and attached to the vertical edges formed by long nanowires on day 3 and 7. RPCs on LNW retain a typical spheroid shape. (e,f) Smooth PCL allows for RPCs to adhere randomly without topographic cues at day 3 and 7.

FIG. 3. Characterization of mRPCs cultured on PCL scaffolds for 7 days. (a-h),(i-p),(q-x) short nanowire, long nanowire and smooth mRPC seeded PCL scaffolds, respectively. (a,i,q) CRX did not show expression. (b,j,r) PKC showed expression only on SNW and LNW. (c,k,s) While Nestin was expressed on SNW, LNW and smooth, (d,l,t) Ki67 was not. (e,m,u) 4D2 was only expressed on SNW. The glial cell marker (f,n) GFAP was expressed on SNW and smooth PCL. (g,o,w) Recoverin was only expressed on LNW and SNW. (h,p,x) nf-200 showed expression on each scaffold. Each image is overlayed with green=GFP mRPCs, red=cy3 bound primary marker and blue=nuclei labeled with Toto 3. Scale:50 μm.

FIG. 4. RT-PCR of RPCs on PCL. RPC expression under standard culture conditions and at day 7 on SNW. Each gene of interest is tested pair-wise: the first lane is baseline expression, the second is day 7 on PCL. Genes that are clearly down regulated after 7 days of culture on PCL were Pax6, Hes5, B3-tubulin, DCX. Partially down-regulated genes included Nestin and Sox2. The primary up-regulated gene was GFAP.

FIG. 5. PCL scaffold delivery of GFP+ mRPCs to C57bl/6 and Rho −/− Mouse retinal explants. (a,b,c) the migration of GFP mRPCs from SNW, LNW, and Smooth PCL scaffolds, respectively, into C57bl/6 retinal explants at day 7. Scaffolds were seeded with ˜2.5×105 day P0 GFP+ mRPCs and allowed to proliferate in vitro for 7 days. Cells migrated into each retinal layer. (d,e) mRPC migration from SNW and LNW PCL into each cellular (nuclear) layer of the Rho −/−, retinal explants. Note that the outer nuclear layer is absent from the 8-10 week Rho −/− retina due to degeneration. (f) Few mRPCs delivered on Smooth PCL appeared to enter the Rho −/− retina. Scale: 100 μm.

FIG. 6. Differentiation and 3D reconstruction of GFP+ mRPCs delivered to a C57bl/6 retinal explant via nanowire PCL scaffolds. (a,b) 20 μm thick explant section reconstructed from successive 1 μm confocal scans showing high levels of mRPC integration from cells delivered by SNW. Also, in the ONL a transplanted RPC shows morphology similar to a young photoreceptor (arrow). (b) PKC (red) double labeling (arrows) of RPC soma and processes from image a) incorporated into the INL of host retina. (c,d) High numbers of RPCs migrated into the GCL (arrows) from LNW. d) Recoverin (red) labeled RPCs (arrows) in the ONL region of host explant. Scale:100 μm.

FIG. 7. GFP+ mRPC Migration, Integration and Differentiation in Host Retina 30 days following Sub-retinal Transplantation. (a,b) Transplanted GFP+ RPC soma migrate into each retinal layer and co-label for GFAP (red). (a) RPC migrated into GCL extend visible processes into IPL (arrow). (b) Smaller RPCs migrated into ONL and appear to have short processes in the OPL region (arrows). (c,d) Transplanted mRPCs integrate into the OPL region of host retina expressing normal levels of NF-200(c) and recoverin (d).

FIG. 8. GFP+ mRPC Migration, Integration and Differentiation in Rho−/− Retina 30 days following Subretinal Transplantation. (a) Transplanted mRPCs migrate into the degenerated Rho−/− ONL and into the preserved INL and GCL. (b) mRPCs migrated into ONL and INL exhibit an early photoreceptor-like morphology (arrows), while RPCs adjacent to the IPL express GFAP. (c) Small RPCs migrated into ONL express GFP and label positively for recoverin (red) (arrows).

FIG. 9 shows a close up of the surface of short nanowire PCL with approximately 2.5 um nano wires.

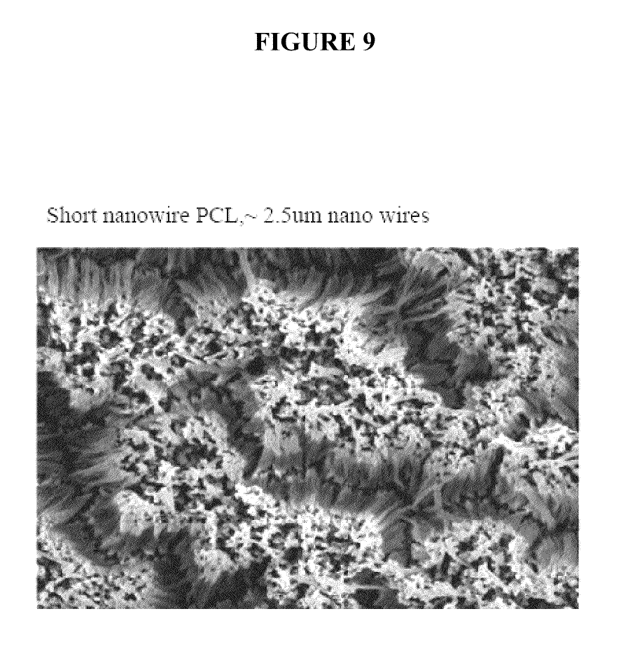

### DESCRIPTION OF THE INVENTION

The present invention provides ocular scaffolds composed of poly (e-caprolactone) configured to be inserted into the sub-retinal space of a subject, as well as methods for treating eye disease (e.g., macular degeneration or age-related macular degeneration) with such scaffolds. The present invention also provides methods of making such scaffolds.

Retinal progenitor cells (RPCs) can be combined with nanostructured polymer scaffolds to generate composite grafts in culture. One strategy for repair of diseased retinal tissue involves implantation of composite grafts of this type in the subretinal space. As described in the Example below, mouse retinal progenitor cells (mRPCs) were cultured on laminin coated novel nanowire poly(e-caprolactone) (PCL) scaffolds and the survival, differentiation and migration of these cells into the retina of C57bl/6 and rhodopsin −/− mouse retinal explants and transplant recipients were analyzed. RPCs were cultured on smooth PCL and both short (2.5 um) and long (27 um) nanowire PCL scaffolds. Scaffolds with adherent mRPCs were then either co-cultured with, or transplanted to, wild type and rhodopsin −/− mouse retina. Robust RPC proliferation on each type of PCL scaffold was observed. Immunohistochemistry revealed that mRPCs cultured on nanowire scaffolds increased expression of mature bipolar and photoreceptor markers. RT-PCR revealed down-regulation of several early progenitor markers. PCL-delivered mRPCs migrated into the retina of both wild type and rhodopsin knockout mice. The results provide evidence that mRPCs proliferate and express mature retinal proteins in response to interactions with nanowire scaffolds. These composite grafts allow for the migration and differentiation of new cells into normal and degenerated retina. Such procedures may be used with human cells to treat human eye diseases, such as macular degeneration.

In certain embodiments, the present invention provides methods for in situ repair of Bruch's membrane, the structure underlying the RPE in the eye and constituting the site of early, fundamental damage in both the exudative (wet) and atrophic (dry) forms of AMD. In certain embodiments, the present invention employs polymeric scaffolds for the treatment of retinal disease through implantation of these structures in the subretinal space of a subject (e.g., human subject). While the present invention is not limited to any particular mechanism, and an understanding of the mechanism is not necessary to practice the invention, it is believed that once the scaffold is positioned in the subretinal space, using standard subretinal surgical procedures, it functions to reconstitute the local microenvironment by effectively serving as a prosthetic Bruch's membrane. While the present invention is not limited to any particular mechanism, it is believed that this allows the host RPE cells to regenerate the RPE monolayer and serves to block ingress by pathological neovascular structures. Again, while the present invention is not limited by any particular mechanism, it is believed that by forming a temporary structural barrier between the RPE and the underlying choriocapillaris, the scaffold serves as a template to allow these host structures to lay down and maintain a new basement membrane structure effectively similar to native Bruch's membrane.

An exemplary material that has found utility for use in the invention is polycaprolactone (PCL). This is based primarily on the ability of PCL to be well tolerated in the subretinal space in a large animal model (pig), together with evidence that a wide range of alternative polymers/materials are not tolerated and result in a giant cell response and/or loss of integrity of the overlying retinal cytoarchitecture. In contrast, host RPE cells are able to grow on the PCL scaffolds and the overlying photoreceptors appear undisturbed despite their juxtaposition to this artificial structure. An example is the use of PCL “nanowire” scaffolds, however, other variations of PCL scaffolds are also well tolerated.

In certain embodiments, the scaffolds of the present invention serve as a platform for cell delivery, e.g., donor RPE cells (including those derived from stem cells) and/or photoreceptors, their precursors, or neural or retinal progenitor cells (NPCs, RPCs), as well as other types of cells. Work conducted during the development of the present invention determined that both brain- and retina-derived progenitor cells will adhere to polymers of various types, with or without protein coating of the scaffolds, and can subsequently be transplanted to the subretinal space of living mammalian recipients.

It has also been determined that the composition of the scaffolds influences the ontogenetic status of adherent immature cells. This can be used to purposefully manipulate phenotypic outcome. For instance, unlike PLGA, PCL tends not to induce the differentiation of co-cultured progenitors cells. Therefore PCL can be used to graft undifferentiated cells or cells previously induced to differentiate down a particular pathway or can be modified to release factors that subsequently influence cellular differentiation, even after implantation in the host.

In other embodiments, the scaffolds of the present invention can be used for sustained local drug delivery to the vicinity of the implant, e.g., growth or neuroprotective factors, cell differentiation factors, pro- or anti-angiogenic factors, pro- or anti-inflammatory agents.

In certain embodiments, the methods, devices, and compositions of the present invention are used the clinical treatment of atrophic (“dry”) AMD as well as the neovascular (“wet”) variants of this disease, including classical and non-classical variants as well as pigment epithelial detachment. Other conditions for which this invention is potentially applicable include hereditary retinal degenerations, such as retinitis pigmentosa, and retinal detachment.

In further embodiments, the polymer scaffolds are combined with Nrl-expressing cells to produce a rod only PCL-RPC composite (2). It has been suggested that the delivery of post-mitotic cells may facilitate differentiation into a selected terminal fate (2, 22). Additionally, PCL nanowire scaffolds can be fabricated to release proteins shown to direct RPCs towards a photoreceptor fate and promote cell survival. The PCL scaffolds of the present invention allow RPCs to proliferate and form a cell dense ultra-thin composite graft for subretinal transplantation. The organized PCL-RPC composite allows for controlled and precisely localizable delivery of cells for the replacement and restoration of retinal tissue destroyed by disease or trauma.

The size of the scaffold implant is generally determined by comparing the clinical assessment of the size of the region of retinal pathology present in a particular patient, with the constraints imposed by surgical feasibility of delivering an implant of a particular size. For example, in degenerations involving the central retina (e.g., age-related macular degeneration), a circular implant of about 1.5 mm diameter (e.g., 1.0-2.5 mm diameter) that approximates the anatomic fovea will frequently be appropriate. In some cases, larger implants may be appropriate, maximally corresponding to the area of posterior retina lying between the temporal vascular arcades (histologic macula, clinical posterior pole) which is an ovoid area of approximately 6.0 mm (vertical)×7.5 mm (horizontal) centered on the fovea. In some instances, it may likewise be appropriate to fashion a polymer scaffold of smaller dimension, as small as about 0.5 mm, to be placed in an area of circumscribed pathology. In addition, it may be of interest to custom fashion implants of irregular shape to suit the patient, for instance to cover areas of pathology while avoiding areas of residual high vision.

The thickness of the polymer component of the implant is generally to be minimized but is generally limited by manufacturing constraints and the physical integrity of the resulting product. It is useful to have an implant under 20 microns in thickness, and 10 microns or less is preferred (e.g. 9 um, 8 um, 7 um, 6 um, 5 um, 4 um, 3 um, 2.5 um, 2 um, 1.5 um, or 1 um).

For use in other retinal diseases, larger implants could be used. These would again be sized to address individual pathology and would be primarily limited in size by surgical constraints related to the need to focally detach that part of the patient's retina for placement of the implant in the subretinal space.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); and C (degrees Centigrade).

### Example 1

### Ocular Scaffolds

A number of advances have resulted from recent efforts to repair retinal tissue damaged by disease. Age-related macular degeneration and retinitis pigmentosa are two examples of diseases in which there is loss of photoreceptor cells. While the adult mammalian retina lacks the ability to spontaneously regenerate, a growing body of evidence supports the hypothesis that retinal tissue can be replaced and some degree of functional recovery regained following the delivery of retinal progenitor cells (RPCs) to the subretinal space (1-2). Subretinally transplanted progenitor cells have the capacity to migrate into the adult retina by following the radially oriented resident glial cells (3). However, studies using subretinal cell injection lose high percentages of RPCs due to cell death and efflux during the transplantation process (1, 4). In recent work, it was demonstrated that the delivery of RPCs on polymer scaffolds results in significantly higher survival of transplanted cells and consequently higher levels of RPC integration (4, herein incorporated by reference in its entirety). To further enhance RPC survival and direct differentiation, this Example implements a novel biodegradable nanostructured poly(e-caprolactone) (PCL) scaffold for cell seeding and subretinal transplantation (5). The PCL scaffold provides a transient structure for high cell number delivery to localized regions of photoreceptor cell loss.

One aspect of embodiments of this PCL scaffold is a topology of vertically oriented nanowires designed to facilitate RPC adhesion and growth (5, herein incorporated by reference in its entirety). The PCL nanowires are formed by hot melt template synthesis with an average diameter of 150-200 nm, and an interwire distance of 20 nm. By varying melt temperature and contact time, nanowire lengths can be specifically tailored. In this Example, two nanowire lengths were examined: short (2.5 μm) and long (27.5 μm). In the in vitro component of this Example, short nanowire (SNW), long nanowire (LNW), and smooth (control) PCL scaffolds were evaluated for their influence on the genetic expression and proliferative capacity of RPCs. Previous studies have shown that polymer scaffold topologies can direct progenitor cell morphology and gene expression (6-8).

A primary objective in this Example was to evaluate the proliferative capacity and gene expression of RPCs seeded on PCL composites in vitro. It was believed that RPC gene expression could be directed towards mature retinal cell types when in contact with the nanowire surface. Secondly, the migration and differentiation of RPCs delivered on PCL scaffolds into normal and degenerative retinal explant models was examined. Finally, RPC-PCL composites were transplanted into the subretinal space of C57bl/6 and Rho −/− mice for one month. Highly organized and concentrated numbers of RPCs delivered on PCL scaffolds in vivo, as well as integration, differentiation and long-term survival of the transplanted cells, were observed.

### Methods

### Mouse Progenitor Cell Isolation and Culture

All experiments were performed according to the Schepens Eye Research Institute Animal Care and Use Committee and the ARVO Statement for the Use of Animals in Ophthalmic and Vision Research. Isolation of RPCs was performed as previously described (4). Briefly, retinas were isolated from postnatal day 1 enhanced green fluorescent protein positive (GFP⁺) transgenic mice (C57BL/6 background). Pooled retina were dissociated by mincing, and digested with 0.1% type 1 collagenase (Sigma-Aldrich; St. Louis, Mo.) for 20 min. The liberated RPCs were passed through a 100 mm mesh filter, centrifuged at 850 rpm for 3 min, re-suspended in culture medium (Neurobasal (NB); Invitrogen-Gibco, Rockville, Md.) containing 2 mM L-glutamine, 100 mg/ml penicillin-streptomycin, 20 ng/ml epidermal growth factor (EGF; Promega, Madison, Wis.) and neural supplement (B27; Invitrogen-Gibco) and plated into culture wells (Multiwell, Becton Dickinson Labware, Franklin Lakes, N.J.). Cells were provided 2 ml of fresh culture medium on alternating days for 2-3 weeks until RPCs were visible as expanding non-adherent spheres. RPC cultures were passaged 1:3 every 7 days.

### Polymer Fabrication

A polymer casting solution was prepared by dissolving PCL in dichloromethane (4% w/v) (Sigma-Aldrich). The PCL solution was cast onto a nanoporous anodized aluminum oxide template using a spin coater (Specialty Coating Systems, Indianapolis, Ind.). The solvent was allowed to evaporate at room temperature. Polymer melts were formed at 130° C. while in contact with the nanoporous template. Nanowire length was tuned as a function of melt time. A melt time of 5 min formed nanowires 2.5 um in length, while a melt time of 60 min formed nanowires 27.5 um in length. The thin-film scaffold of vertically aligned nanowires was released by etching the template in a dilute sodium hydroxide solution, and allowed to air dry at room temperature. Smooth control PCL scaffolds were fabricated on an electrochemically polished silicon wafer using a spin-cast/solvent evaporation method.

### Polymer Preparation, Cell Seeding and Culture

PCL nanowire and smooth scaffolds (4×4 mm) were incubated in 70% ethanol for 24 h and rinsed 3 times with Phosphate Buffered Saline Solution (PBS). PCL scaffolds were placed into single wells of 12 well culture plates and incubated in 50-100 μg/ml mouse laminin (Sigma) in PBS for 12 h to facilitate subsequent adhesion of RPCs in culture. Polymers were then rinsed 3 times with PBS and transferred to 0.4 μm pore culture well inserts (Falcon) in 12 well plates. Scaffolds were then submerged in 1 ml of NB and incubated for 1 hr at 37° C. Cultured GFP⁺ RPCs were dissociated into single cell suspensions and 100 μl (4×10⁵ cells) seeded onto each laminin-coated PCL membrane. The total volume of each well was brought to 2 ml with additional NB media and RPCs were allowed to proliferate on the polymers for 7 days.

### SEM

Cell morphology on smooth, SNW, and LNW PCL substrates was examined using SEM after 1, 3, and 7 days of culture. Prior to imaging, the cells were fixed and dehydrated. Each sample was rinsed twice in PBS and then soaked in a primary fixative of 3% glutaraldehyde, 0.1M of sodium cacodylate, and 0.1M sucrose for 72 hours. The surfaces were subjected to 2 five-minute washes with a buffer containing 0.1M sodium cacodylate and 0.1M sucrose. The cells were then dehydrated by replacing the buffer with increasing concentrations of ethanol for ten minutes each. The cells were dried by replacing ethanol with hexamethyldisilazane (HMDS) (Polysciences) for 10 minutes, and subsequently air dried for 30 minutes. After mounting, the samples were sputter-coated with a 15 nm layer of gold-palladium at a current of 20 mA and a pressure of 0.05 mbar for 45 s. SEM imaging was conducted on a FEI XL30 Sirion Scanning Electron Microscope at 5 kV.

### Cell Growth and Proliferation on PCL

Expansion of GFP⁺ mRPCs was analyzed on SNW, LNW and smooth PCL. To establish a standard mRPC population curve, total mRPC GFP⁺ signals were detected in populations from 1×10³−1.5×10⁵ in 96 well plates (n=4) using a Tecan, Genios microplate reader. A 1.0×1.0 mm piece of each PCL subtype was then seeded with mRPCs and cultured for 7 days. Total GFP emissions from RPCs on each polymer type were taken at days 1, 3, and 7 under identical conditions. The RPC-polymer signals and standard population curve signals were then correlated to establish polymer cell density. The composites were also imaged at 10× magnification at days 1, 3, and 7. After the initial seeding of cells a Spot ISA-CE camera (Diagnostic Instruments, Sterling Heights, Calif.) attached to a Nikon Eclipse TE800 microscope was used to visualize cell proliferation across each PCL sub-type surface.

### Immunohistochemistry

After culturing RPCs for 7 days, RPC-polymer composites were rinsed 3 times with PBS (warmed to 37° C.) and fixed in 4% paraformaldehyde for 1 h, cryoprotected first in 10% sucrose for 12 h and then in 30% sucrose for 12 h. Cryoprotected composites were frozen in Optimal Cutting Temperature Compound (Sakura Finetek, Torrence, Calif.) at −20° C. and cut into 20 μm sections using a Minotome Plus (Triangle Biomedical Sciences, Durham, N.C.). All samples were rinsed 3×10 min in PBS and then blocked and permeabilized in PBS containing 10% goat serum, 1% BSA, and 0.1% Triton-x for 2 h. Samples were incubated with primary antibodies using a dilution of 1:200 for CRX (Santa Cruz), 1:500 for PKC (Sigma), 1:400 for Nestin (BD Biosciences), 1:100 for Ki67 (Sigma), 1:200 for 4D2 (a gift from Prof. Robert Molday, University of British Columbia, Canada), 1:200 for GFAP (Zymed), 1:100 for Recoverin (Abcam), and 1:1000 for NF-200 (Sigma) in blocking buffer for 12 h at 4° C. Samples were then rinsed 3×10 min in PBS and incubated with a Cy3-labeled secondary antibody 1:800 (Zymed) and Toto-3 (Molecular Perobes) nuclear stain for 2 h at room temperature. Finally, samples were rinsed 3×10 min in PBS and sealed in mounting medium (Vector Laboratories) for imaging using a Leica TCS SP2 confocal microscope.

### Reverse Transcription-Polymerase Chain Reaction

Total RNA was extracted from cultured cells using the RNeasy Mini kit according to the manufacturer's instructions (Qiagen, Calif., USA) followed by in column treatment with DNase I (Qiagen, Calif., USA). Reverse transcription was performed with Omniscriptase Reverse Transcriptase (Qiagen, Calif., USA) and random primers (Sigma, Mo., USA). Amplification of β-actin served as the internal control. The primers for RT-PCR are shown in Table 1. Amplification conditions were 40 sec/94° C., 40 sec/55° C., 1 min/72° C. for 35 cycles.

TABLE 1
List of primers for RT-PCR

| | | |
|---|---|---|
| Gene | Primer sequence (5′-3′) | Product size (bp) |

| | | | |
|---|---|---|---|
| Nestin | F: AACTGGCACACCTCAAGATGT | (SEQ ID NO: 1) | 235 |
| | R: TCAAGGGTATTAGGCAAGGGG | (SEQ ID NO: 2) | |
| Sox2 | F: CACAACTCGGAGATCAGCAA | (SEQ ID NO: 3) | 190 |
| | R: CTCCGGGAAGCGTGTACTTA | (SEQ ID NO: 4) | |
| Pax6 | F: AGTGAATGGGCGGAGTTATG | (SEQ ID NO: 5) | 132 |
| | R: ACTTGGACGGGAACTGACAC | (SEQ ID NO: 6) | |
| Hes1 | F: CCCACCTCTCTCTTCTGACG | (SEQ ID NO: 7) | 185 |
| | R: AGGCGCAATCCAATATGAAC | (SEQ ID NO: 8) | |
| Hes5 | F: CACCGGGGGTTCTATGATATT | (SEQ ID NO: 9) | 180 |
| | R: CAGGCTGAGTGCTTTCCTATG | (SEQ ID NO: 10) | |
| Ki-67 | F: CAGTACTCGGAATGCAGCAA | (SEQ ID NO: 11) | 170 |
| | R: CAGTCTTCAGGGGCTCTGTC | (SEQ ID NO: 12) | |
| β-tubulin III | F: CGAGACCTACTGCATCGACA | (SEQ ID NO: 13) | 152 |
| | R: CATTGAGCTGACCAGGGAAT | (SEQ ID NO: 14) | |
| Dcx | F: ATGCAGTTGTCCCTCCATTC | (SEQ ID NO: 15) | 182 |
| | R: ATGCCACCAAGTTGTCATCA | (SEQ ID NO: 16) | |
| Recoverin | F: ATGGGGAATAGCAAGAGCGG | (SEQ ID NO: 17) | 179 |
| | R: GAGTCCGGGAAAAACTTGGAATA | (SEQ ID NO: 18) | |
| Rhodopsin | F: TCACCACCACCCTCTACACA | (SEQ ID ND: 19) | 216 |
| | R: TGATCCAGGTGAAGACCACA | (SEQ ID NO: 20) | |
| GFAP | F: AGAAAACCGCATCACCATTC | (SEQ ID NO: 21) | 184 |
| | R: TCACATCACCACGTCCTTGT | (SEQ ID NO: 22) | |
| β-actin | F: AGCCATGTACGTAGCCATCC | (SEQ ID NO: 23) | 228 |
| | R: CTCTCAGCTGTGGTGGTGAA | (SEQ ID NO: 24 | |

### Retinal Explant Culture

C57bl/6 (n=3) and rhodopsin knockout (Rho−/−) (n=3) mice were euthanized and their eyes enucleated immediately and placed in ice cold PBS. The anterior portion of each eye was removed along with vitreous. Four radial cuts were made into the posterior eyecup and each quadrant flattened sclera side down. The flattened eyecup was then cut into four separate pieces (˜2.0×2.0 mm) and transferred to a 0.4 μm culture well insert, ganglion side down, and sclera removed. Culture well inserts containing retina were placed into 6 well culture plates. 2 mls of NB were added to each culture well. Onto each retinal explant a 7 day cultured RPC-PCL (2.0×1.0 mm) composite was placed. Three SNW, LNW and smooth RPC seeded PCL constructs (n=18) were added to both C57bl/6 and Rho−/− explants and co-cultured for one week in NB at 37° C.

### Subretinal Transplantation Surgery

Transplantation surgeries were performed as previously described (4). Briefly, SNW and LNW PCL scaffolds with adherent RPCs were cut into 1.0×0.5 mm sections using a sterile scalpel in preparation for transplantation. Mice were placed under general anesthesia with an intraperitoneal injection of ketamine (5 mg/kg) and xylazine (10 mg/kg) and the pupil dilated with 1% tropicamide, topically applied. Proparacaine (Akorn), a topical anesthetic, was applied to the eye. Mice were placed on a warm heating blanket for surgery. Silk sutire (8-0) was used to retract the eyelid and the globe was stabilized for surgery using a single 11-0 conjuctival suture. An incision (0.5-1.0 mm) was made in the lateral posterior sclera using a Sharpoint 5.0 mm blade scalpel (Fine Science Tools, Reading, Pa.) PCL-RPC composites were inserted through the sclerotomy into the subretinal space using #5 Dumont forceps (Fine Science Tools). A single eye from each C57BL/6 wild-type mouse (n=10) and Rho −/− mouse (n=10) received a subretinal transplant. The scleral incision was closed with an 11-0 nylon suture and all other sutures were removed. Additional proparacain was applied and mice were allowed to recover. Transplants remained in the subretinal space for one month.

### Histologic Preparation of Transplanted Tissue

C57BL/6 mice that received composite grafts were sacrificed after 4 weeks. Engrafted eyes were enucleated, immersion fixed in 4% paraformaldehyde, rinsed 3 times in PBS and cryoprotected in 10% then 30% sucrose for 12 h each at 4° C. Eyes were then placed in a cryomold containing optimum cutting temperature (O.C.T) compound (ProSciTech) and then frozen on dry ice and cryosectioned at 20 μm.

### Results

### Polymer Preparation, Cell Seeding and Culture

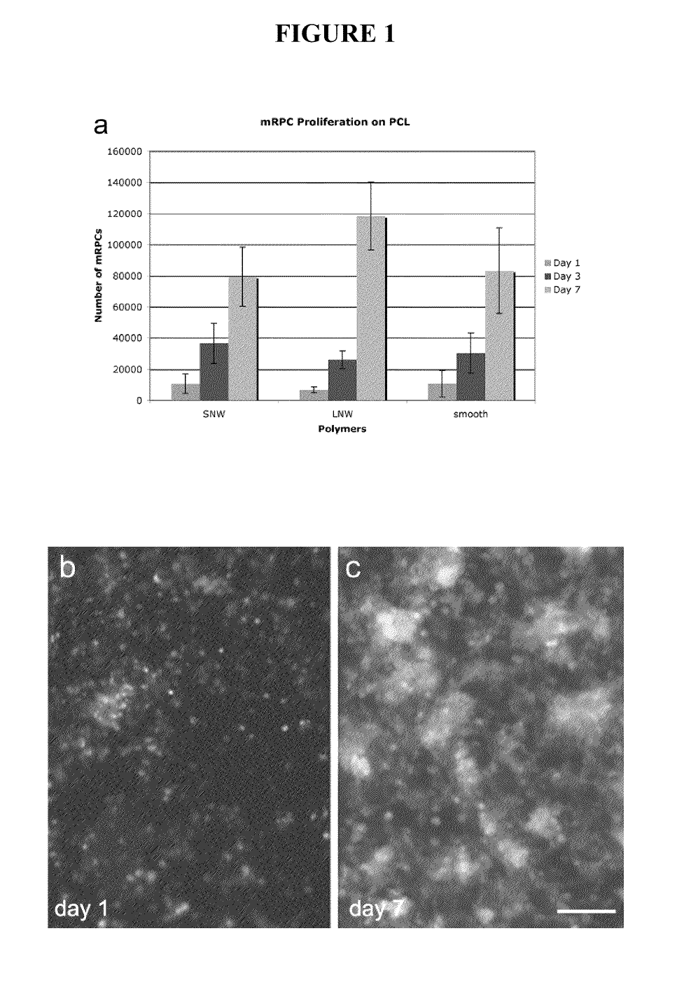

RPC survival and proliferation were similar when cultured on each type of PCL scaffold studied (FIG. 1). After seeding 4×10⁵ cells into culture wells containing 1.0×1.0 mm PCL scaffolds, a similar number of cells had adhered to each topology type as revealed by averaged GFP⁺ fluorescence intensities (FIG. 1A). Cell numbers increased steadily for the remaining seven days in culture. At days 1, 3, and 7, the averaged (n=3) number of cells were SNW: 6688, 36478, 95542, LNW: 6799, 26044, 118389, and Smooth: 3973, 30217, 83205, respectively. RPC densities at day 1 and increased cell density through at day 7 can be seen in FIGS. 1B and 1C, respectively. Based on initial seeding densities, the proliferation rate correlates well with the 24 hour cell cycle of proliferating mRPCs.

Scanning Electron Microscopy of mRPC Seeded Scaffolds

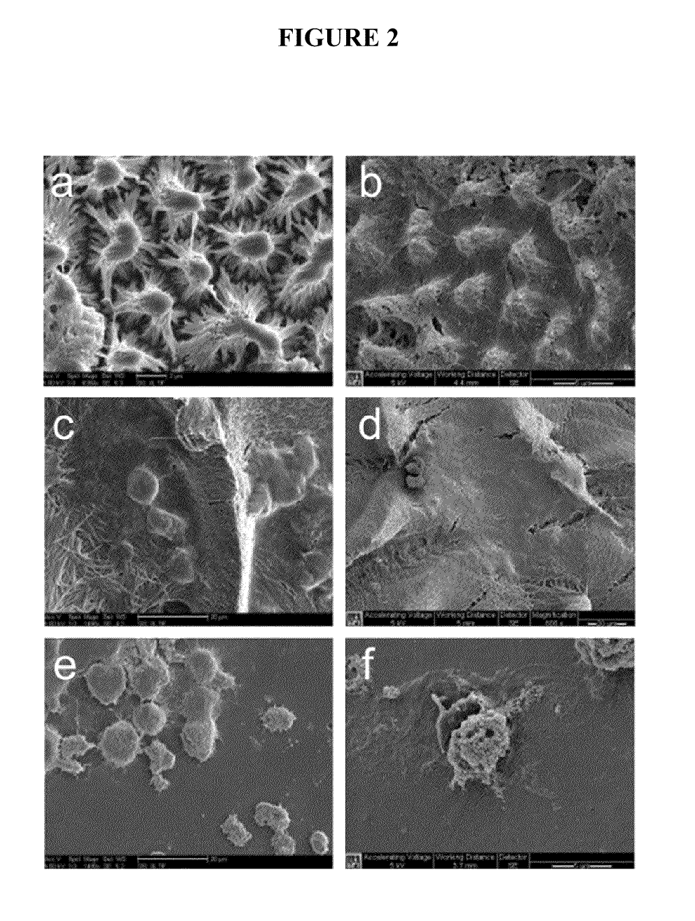

RPCs cultured at low-densities for SEM imaging on nanowire PCL exhibited apparent polymer topology attachment patterns and/or morphologic changes at 3 and 7 days (FIG. 2). The most pronounced morphologic changes occurred in RPCs cultured on SNW PCL at days 3 and 7 (FIGS. 2A and 2B). On SNW individual RPCs adhered to clustered tips of 2.5 μm nanowires and spread fan-like processes (˜20 nm) out to neighboring cells, creating apparent cell-to-cell contacts. RPCs cultured on LNW PCL formed small clusters on the underside of wave-like aggregates of the 27.5 μm nanowires and maintained their spheroid shape at days 3 and 7 (FIGS. 2C and 2D). RPCs seeded onto smooth PCL adhered at random intervals to each surface and showed no distinctive morphologic changes at either day 3 or 7 and exhibited no alignment with specific surface regions (FIGS. 2E and 2F).

### Immunohistochemistry

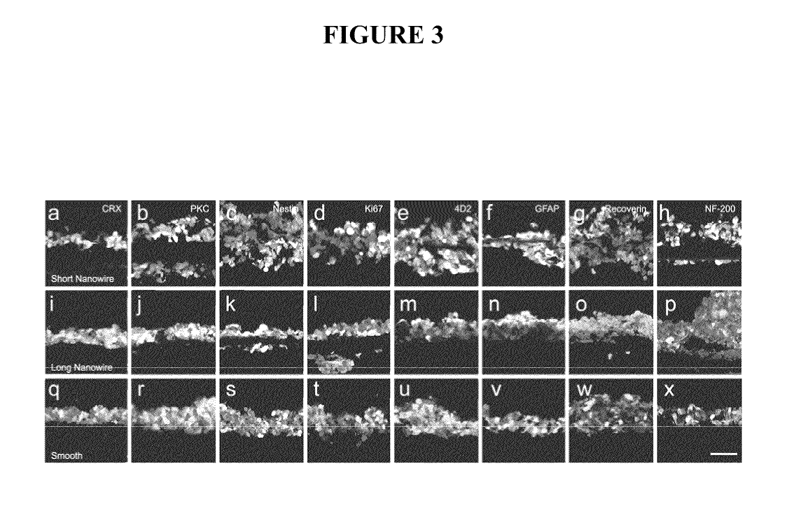

Immunohistochemical analysis of mRPCs cultured on PCL revealed that scaffold topology influenced protein expression levels (FIG. 3). The markers used to evaluate mRPC differentiation included the early photoreceptor marker CRX, the bipolar cell marker PKC, the neural progenitor marker nestin, the active cell cycle marker Ki67, the mature photoreceptor marker 4D2, the glial cell marker GFAP, the photoreceptor marker recoverin, and the neural filament maker nf-200. On each sub-type of PCL polymer mRPCs consistently labeled positively for nestin and nf-200, indicating the presence of undifferentiated cell populations. Mouse RPCs cultured on SNW and LNW nanowire scaffolds demonstrated evidence of differentiation including increased expression of PKC and recoverin. Smooth PCL produced no detectable changes in mRPC expression of mature retinal neural markers. Interestingly, SNW topology induced increases in the rod photoreceptor protein rhodopsin, as well as recoverin and PKC.

### RT-PCR

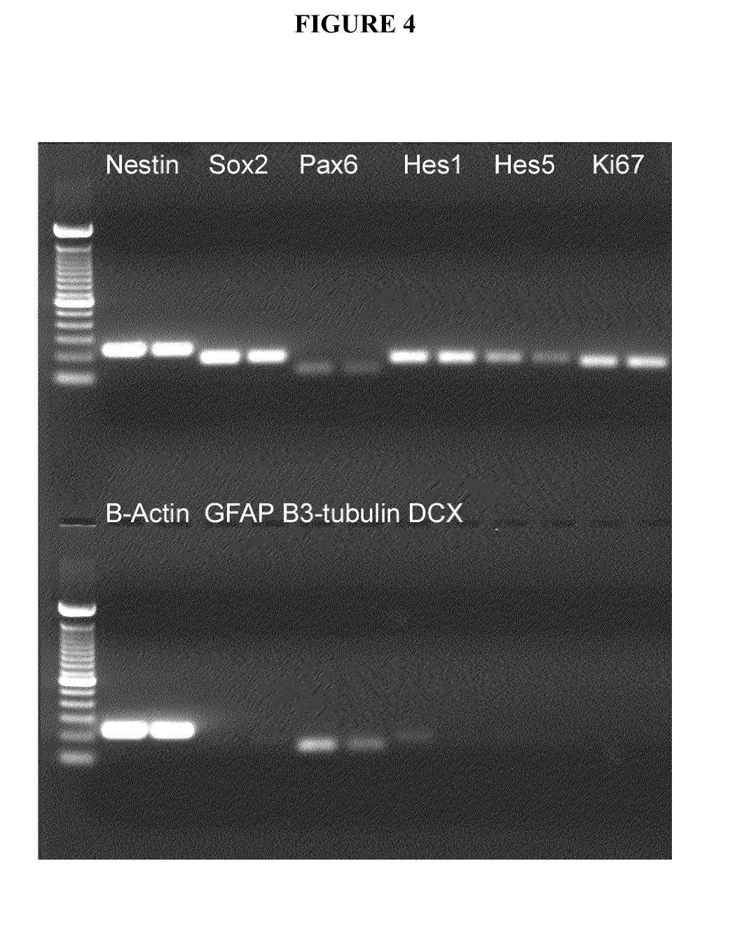

Analysis of RNA synthesis in RPCs using RT-PCR revealed marked down-regulation of Pax6, Hes1, B3-tubulin, DCX and partial down-regulation of nestin and Sox2 (FIG. 4). GFAP was up-regulated. Decreases in the expression levels of Pax6, Hes 1, nestin and Sox2 suggest that the immature RPCs had begun undergoing differentiation toward more mature states while co-cultured on the polymer scaffolds.

Migration and Differentiation of mRPCs in Retinal Explants

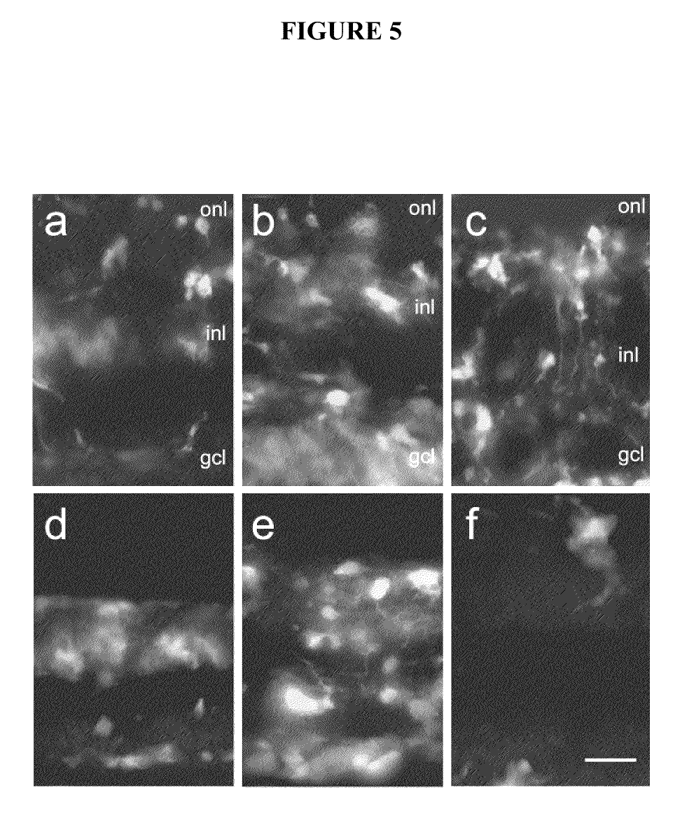

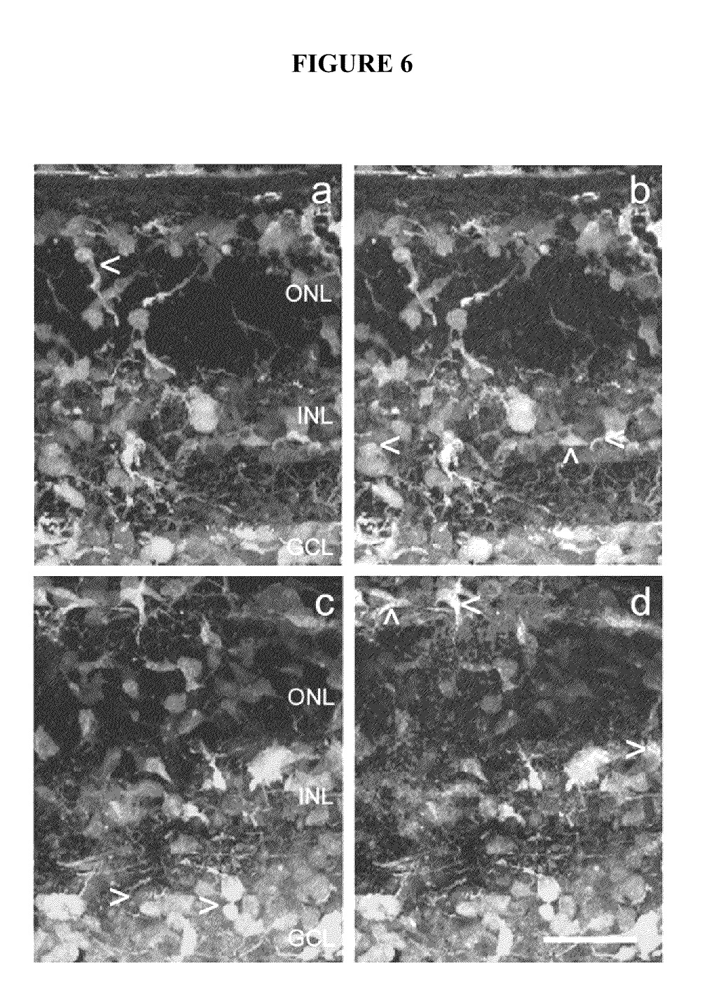

At 1 week, RPC-PCL composites of each topology type cultured on either C57bl/6 or Rho−/− retinal explants allowed for RPC migration into each retinal layer and expression of location-appropriate, fate-specific markers (FIG. 5). Both C57bl/6 and Rho−/− mouse retinal explants proved permissive environments for the migration of progenitor cells to specific retinal layers. Both SNW and LNW RPC composites resulted in high levels of migration into the inner nuclear and ganglion cell layers (INL, GCL) of the Rho−/− explants. Smooth PCL RPC composites did not provide for integration into the Rho−/− model. Widespread integration of RPCs into C57bl/6 retinal lamina was seen (FIG. 5A-C). The soma of integrated RPCs aligned with host nuclear layers, from which they extended processes toward and into each plexiform layer. RPC-SNW and LNW composites cultured on explants developed into profiles similar to glial, bipolar and rod phenotypes. The migration and differentiation of RPCs was not significantly different between SNW and LNW explants. Three dimensional views of RPC integration from SNW and LNW composites into 20 μm thick explants reconstructed from 1 μm confocal scans can be seen in FIGS. 6A-B and 6C-D, respectively. The expression of PKC and recoverin were seen in RPCs that migrated into the outer and inner plexiform (OPL, IPL) layers, respectively (FIGS. 6B and 6D).

### RPC Migration and Differentiation Following Subretinal Transplantation,

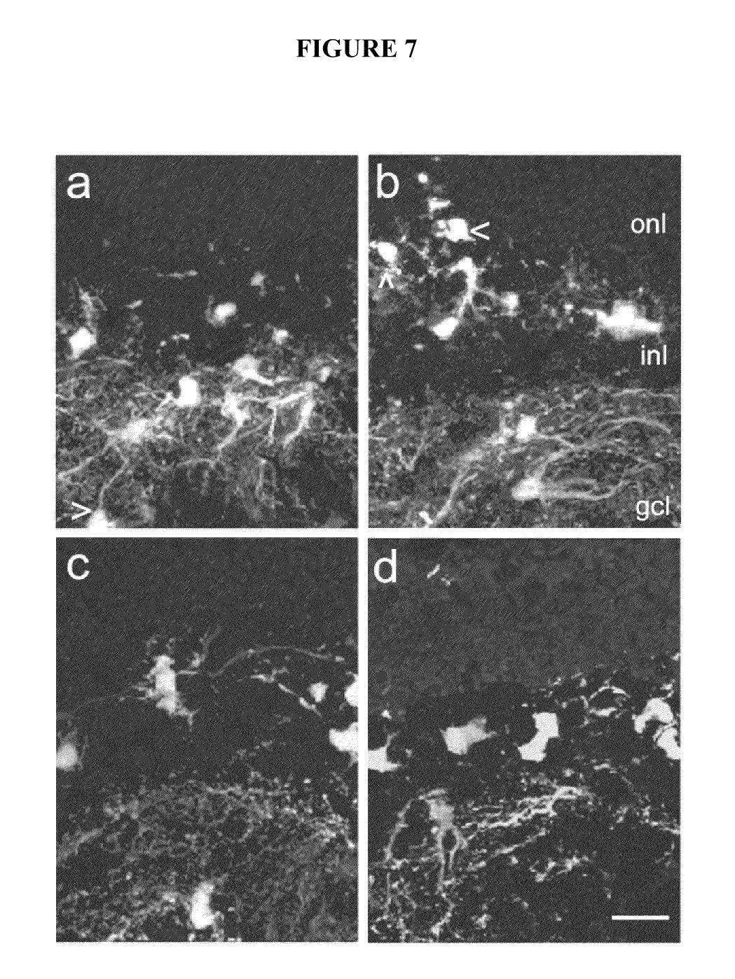

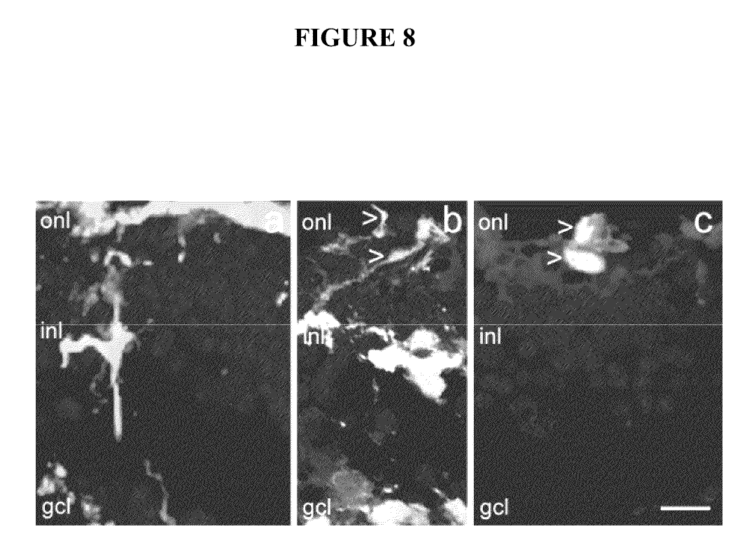

Based on lower RPC proliferation and migration into explants, smooth PCL was not transplanted in vivo. After one month in the subretinal space of C57bl/6 and Rho−/− mice, mRPCs grafted on LNW and SNW scaffolds had migrated into specific retinal laminae, extended processes and differentiated morphologically (FIGS. 7 and 8). In normal C57bl/6 mice, many RPCs migrated to the INL/IPL region and adopted a morphology similar to glial or amacrine cells with processes, extending 10-50 μm. RPCs that migrated to the IPL showed expression of GFAP (FIGS. 7A and B). Projections from RPC soma integrated into the IPL, extended through the IPL and occasionally reached into both the IPL and GCL layers. RPCs which migrated into the outer nuclear and outer plexiform layers (FIGS. 7C and D), (ONL, OPL) extended shorter (˜5-10 μm) processes remaining in the ONL or extending into the OPL. RPCs that migrated into the outer retina appeared to connect in regions with cells expressing either PKC or recoverin, respectively (FIGS. 7C and D). A high number of RPCs were seen to have migrated into host retinal tissue directly adjacent to the site of transplantation. In Rho−/− recipients, RPCs migrated into the degenerated ONL and into the remaining INL and GCL (FIG. 8A). A number of mRPCs that had migrated into the Rho−/− retina ONL and INL developed an apparent cell polarity with early photoreceptor-like morphology, while mRPCs adjacent to the IPL expressed GFAP (FIG. 8B). Unique to the Rho−/− recipients, small diameter (˜10 μm) RPCs migrated into the ONL and expressed recoverin (FIG. 8C). The area of host retinal integration was approximately 0.3×0.8 μm, similar to the transplant size. Highly localized delivery of RPCs incorporated into the host retinal laminae across the area of the transplant was observed.

### Discussion

In this Example, it was shown that RPCs can be co-cultured with PCL nanowire substrates and that these scaffolds are biologically compatible with RPCs, as evidenced by cell adhesion and sustained proliferation. This work complements earlier studies which analyzed the biocompatibility of micro-patterned polymer substrates both in vitro and in vivo (4, 7, 9, all of which are herein incorporated by reference). To avoid physical distortion and metabolic impairment of the outer retina, implantation in the subretinal space puts a premium on the thinness of the scaffold used. The nanowire scaffolds presented here represent the thinnest and most intricately patterned polymer substrates that have been used for RPC subretinal transplantation to date.

The basement PCL sheet from which both short and long nanowires project is on average 6.00±0.70 μm thick. The thin-film structure of nanowire PCL offers at least two significant advantages for subretinal transplantation. Firstly, RPC-seeded PCL scaffolds can be placed into the subretinal space with minimal disturbance of surrounding tissue. Secondly, PCL is highly permeable, allowing for the passage of physiologically significant molecules, as well as predictable degradation of the scaffold itself. After 7 weeks in saline, nanowire features are completely degraded (5). The biodegradation of PCL occurs gradually from its surfaces and shows no pathologic increases in local acidity as seen in the bulk degradation of polymers composed of higher molecular weight PGLA (10). The nano-scale architecture and degradation rate of PCL nanowire scaffolds are well suited for subretinal RPC delivery.

Polymer substrates for tissue engineering with either nanowire or micro-patterned porous three-dimensional structures have been shown to enhance progenitor cell adhesion (7, 9). In a recent study it was demonstrated that poly(methyl methacrylate) (PMMA) scaffolds micro-machined to contain through pores provided greater RPC adhesion during transplantation than a non-structured PMMA control (9). For the purpose of RPC culture and eventual delivery of RPCs into the subretinal space an optimal polymer scaffold should provide either surface or internal cavities to protect cell-to-polymer contacts from mechanical and shearing forces. The surface patterning of PCL nanowire scaffolds provide niches for secure and organized cell adhesion.

Combining cells with polymer substrates to engineer implants directed at repairing retinal tissue requires attention to the interacting properties of the particular cell type and the chosen polymer. In the present Example, it was important to consider the relationship between the response properties of the selected RPC population and the microenvironment of the PCL nanowire scaffolds, particularly with respect to how this might influence retinal cell fates. The RPCs used in this study were isolated from GFP⁺ C57BL/6 mice at post-natal day 0 (P0), a developmental time shown to produce primarily rod, bipolar and Mueller cells (11-13). The transient expression of Notch and yan, receptors by P0 mRPCs provide examples of known pathways capable of influencing cell fate in response to exogenous signaling. In a further example, in the presence of ciliary neurotrophic factor (CNTF), which is produced by the developing retina, higher numbers of P0 RPCs can be driven to express opsin (12). After time in culture, P0 RPCs not expressing opsin and exposed to CNTF tend to differentiate toward a bipolar cell fate (14). Under the proliferation conditions used in this Example, RPCs were incubated in elevated levels (20 ng/ml) of epidermal growth factor (EGF) to maintain mitogenic activity. According to one report, P0 RPCs transiently express the EGF receptor (EGFR) and proliferate in response to EGF via a Notch signaling pathway (15). It has also been reported that exposure to EGF has the potential to over-ride intrinsic fate cues of late progenitors and drive differentiation towards a glial fate (15-16). Earlier studies demonstrated that PLGA scaffolds tend to sequester EGF from the surrounding medium and the PCL material used in the current study might potentially behave in a similar manner. In this way, GFAP expression by RPCs on SNW in vitro might result from decreased availability of EGF and hence the influence of diminished EGF signaling on cell competence. Another possibility is that treatment of scaffolds with the substrate laminin, used to promote cellular adherence for transplantation, might also have contributed to the observed changes in cellular behavior.

The morphologic changes of RPCs that occurred in response to SNW scaffold architecture involved the anchoring of cell soma to aggregated nanowire tips with extension of lamellipodia-like structures toward adjacent cells. The RPCs made apparent contacts with one another forming uniform monolayers across aggregated nanowire bundles. This type of cell morphology across a polymer surface has been referred to as an “adhesion plaque” and serves to strengthen cell-to-substratum attachment (17). In addition to geometric constraints conferred by the fine structure of the nanowire scaffolds, the morphology of co-cultured RPCs is likely influenced by any changes in cellular phenotype occurring under these circumstances, as discussed in previous studies (7, 18). Taken together, the gene expression patterns and substrate-directed morphologies indicate a trend toward more mature phenotypes for mRPCs cultured on laminin-treated PCL nanowire substrates.

The characterization of cycling uncommitted multipotent RPCs is challenged by the tendency of these cells to express a range of different neural and glial fate-related transcripts (19). Individual multipotent RPCs of the same type exhibit transient changes in molecular heterogeneity at different time points. After terminal mitosis, non-fate specific markers are down-regulated while selected fate markers are more strongly expressed. Even after RPCs have exited the mitotic cycle, they retain a level of plasticity and can change expression patterns and redirect fate (20). In this study, mitogenic sub-populations of RPCs interacting with PCL nanowires could be seen to up-regulate fate-specific markers. Nevertheless, these results indicate a trend toward a differentiated state rather than clear evidence of terminal differentiation. The nanowire surface appears to be capable of at least partially modifying the growth kinetics, morphology and expression patterns of adhering progenitor cells. Co-culture of RPC-containing polymers with retinal explants resulted in migration of the progenitor cells into each retinal layer. Of the markers evaluated, the transplanted cells reacted for recoverin and PKC expression. The morphology of the migrated cells resembled glial and neural subtypes appropriate to their region of laminar integration. The in vivo subretinally transplanted RPCs also integrated into each lamina with a preference for IPL and GCL layers. The majority of cells labeled for GFAP expression. This finding may be the result of the developmental potential of the selected RPC population for differentiation towards a glial fate, and/or partially influenced by EGF exposure as discussed above (15, 21).

In terms of transplantation, based on the number (˜100,000) of RPCs attached to 1.0×1.0 mm pieces of SNW and LNW at day 7, we can predict that approximately 50,000 RPCs were delivered on each 0.5×1.0 mm graft that was transplanted. This level of cell delivery was sufficient to achieve direct migration and integration of RPCs from the scaffold into regions of the host retina adjacent to the transplantation site. As such, delivering pre-determined numbers of RPCs to a specific region of the retina damaged by disease or injury may be an approach to retinal tissue repair (4).

### REFERENCES

All of the following references are herein incorporated by reference in their entireties as if full set forth herein.
(1) Klassen H J, Ng T F, Kurimoto Y, Kirov I, Shatos M, Coffey P, Young M J. (2004) Multipotent retinal progenitors express developmental markers, differentiate into retinal neurons, and preserve light-mediated behavior. Invest Opthalmol Vis Sci. November; 45(11):4167-73.
(2) MacLaren R E, Pearson R A, MacNeil A, Douglas R H, Salt T E, Akimoto M, Swaroop A, Sowden J C, Ali R R. (2006) Retinal repair by transplantation of photoreceptor precursors. Nature. November 9; 444(7116):203-7.
(3) Wojciechowski A B, Englund U, Lundberg C, Warfvinge K. (2004) Survival and long distance migration of brain-derived precursor cells transplanted to adult rat retina. Stem Cells. 22(1):27-38.
(4) Tomita M, Lavik E, Klassen H, Zahir T, Langer R, Young M J. (2005) Biodegradable polymer composite grafts promote the survival and differentiation of retinal progenitor cells. Stem Cells. November-December; 23(10):1579-88.
(5) Tao S L, Desai T A. (2007) Aligned arrays of biodegradable poly(epsilon-caprolactone) nanowires and nanofibers by template synthesis. Nano Lett. June; 7(6):695-701.
(6) Recknor J B, Sakaguchi D S, Mallapragada S K. Growth and differentiation of astrocytes and neural progenitor cells on micropatterned polymer films. (2005) Ann N Y Acad Sci. May; 1049:24-7.
(7) Recknor J B, Sakaguchi D S, Mallapragada S K. (2006) Directed growth and selective differentiation of neural progenitor cells on micropatterned polymer substrates. Biomaterials. August; 27(22):4098-108.
(8) Miller C, Jeftinija S, Mallapragada S. Synergistic effects of physical and chemical guidance cues on neurite alignment and outgrowth on biodegradable polymer substrates. (2002) Tissue Eng. July; 8(3):367-78.
(9) Tao S, Young C, Redenti S, Zhang Y, Klassen H, Desai T, Young M J. (2007) Survival, migration and differentiation of retinal progenitor cells transplanted on micro-machined poly(methyl methacrylate) scaffolds to the subretinal space. Lab Chip. June; 7(6):695-701.
(10) Grayson A C, Voskerician G, Lynn A, Anderson J M, Cima M J, Langer R. (2004) Differential degradation rates in vivo and in vitro of biocompatible poly(lactic acid) and poly(glycolic acid) homo- and co-polymers for a polymeric drug-delivery microchip. J Biomater Sci Polym Ed. 15(10):1281-304.
(11) Rapaport D H, Wong L L, Wood E D, Yasumura D, LaVail M M. (2004) Timing and topography of cell genesis in the rat retina. J Comp Neurol. June 21; 474(2):304-24.
(12) Cepko C L, Austin C P, Yang X, Alexiades M, Ezzeddine D. (1996) Cell fate determination in the vertebrate retina. Proc Natl Acad Sci USA. January 23; 93(2):589-95.
(13) Livesey F J, Cepko C L. Vertebrate neural cell-fate determination: lessons from the retina. (2001) Nat Rev Neurosci February; 2(2):109-18. Review.
(14) Zahir T, Klassen H, Young M J. (2005) Effects of ciliary neurotrophic factor on differentiation of late retinal progenitor cells. Stem Cells. March; 23(3):424-32.
(15) James J, Das A V, Rahnenfuhrer J, Ahmad I. (2004) Cellular and molecular characterization of early and late retinal stem cells/progenitors: differential regulation of proliferation and context dependent role of Notch signaling. J Neurobiol. December; 61(3):359-76.
(16) Irvin D K, Dhaka A, Hicks C, Weinmaster G, Kornblum H I. (2003) Extrinsic and intrinsic factors governing cell fate in cortical progenitor cultures. Dev Neurosci. March-August; 25 (2-4):162-72.
(17) Lu L, Yaszemski M J, Mikos A G. (2001) Retinal pigment epithelium engineering using synthetic biodegradable polymers. Biomaterials. December; 22(24):3345-55. Review.
(18) Lavik E B, Klassen H, Warfvinge K, Langer R, Young M J. (2005) Fabrication of degradable polymer scaffolds to direct the integration and differentiation of retinal progenitors. Biomaterials. June; 26(16):3187-96.
(19) Trimarchi J M, Stadler M B, Roska B, Billings N, Sun B, Bartch B, Cepko C L. (2007) Molecular heterogeneity of developing retinal ganglion and amacrine cells revealed through single cell gene expression profiling. J Comp Neurol June 20; 502(6):1047-65.
(20) Belliveau M J, Young T L, Cepko C L. (2000) Late retinal progenitor cells show intrinsic limitations in the production of cell types and the kinetics of opsin synthesis. J Neurosci. March 15; 20(6):2247-54.
(21) Klassen H, Sakaguchi D S, Young M J. Stem cells and retinal repair. (2004) Prog Retin Eye Res. March; 23(2):149-81.
(22) Canola K, Angenieux B, Tekaya M, Quiambao A, Naash M I, Munier F L, Schorderet D F, Arsenijevic Y. (2007) Retinal stem cells transplanted into models of late stages of retinitis pigmentosa preferentially adopt a glial or a retinal ganglion cell fate. Invest Opthalmol Vis Sci. January; 48(1):446-54.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry and molecular biology or related fields are intended to be within the scope of the following claims.

We claim:

1. A device comprising a scaffold configured to be inserted into the sub-retinal space of a subject, wherein said scaffold comprises poly(e-caprolactone).

2. The device of claim 1, wherein said scaffold is formed from nanowires, wherein said nanowires comprise said poly(e-caprolactone).

3. The device of claim 1, further comprising donor cells, wherein said scaffold is at least partially coated with said donor cells.

4. The device of claim 1, wherein said donor cells are selected from: RPE cells, stem cells, photoreceptors, precursors, neural or retinal progenitor cells (NPCs and RPCs).

5. The device of claim 1, wherein said device further comprises a protein coating, and wherein said scaffold is coated with said protein coating.

6. The device of claim 6, wherein said protein coating comprises laminin.

7. The device of claim 1, wherein said scaffold is further configured to serve as a prosthetic Bruch's membrane.

8. The device of claim 1, wherein said scaffold is between about 4-40 mm in length, and about 4-40 in width.

9. The device of claim 1, wherein said scaffold has a thickness of about 5-7 um.

10. A method of making a device for insertion into the sub-retinal space of a subject comprising: contacting a scaffold with donor cells such that said scaffold is at least partially coated by said donor cells, wherein said scaffold is configured to be inserted into the sub-retinal space of a subject and comprises poly(e-caprolactone).

11. The method of claim 10, wherein said scaffold is formed from nanowires, wherein said nanowires comprise said poly(e-caprolactone).

12. The method of claim 10, wherein said donor cells are selected from: RPE cells, stem cells, photoreceptors, precursors, neural or retinal progenitor cells (NPCs and RPCs).

13. The method of claim 10, wherein said scaffold further comprises a protein coating, and wherein said scaffold is coated with said protein coating.

14. The method of claim 13, wherein said protein coating comprises laminin.

15. The method of claim 10, wherein said scaffold is further configured to serve as a prosthetic Bruch's membrane.

16. The method of claim 10, wherein said scaffold is between about 4-40 mm in length, and about 4-40 in width.

17. The method of claim 10, wherein said scaffold has a thickness of about 5-7 um.
